(19) United States
(12) Patent Application Publication
(10) Pub. No.: US 2026/0248470 A1
(43) Pub. Date: Aug. 27, 2026

(54) APPARATUS FOR POSITIONING A PATIENT WITH PIVOTABLY MOUNTED HARDWARE MODULE

(71) Applicant: Siemens Healthineers AG, Forchheim (DE)

(72) Inventors: Klaus HRUSCHKA, Erbendorf (DE); Berthold BAUMANN, Kastl (DE); Daniel EIGNER, Erbendorf (DE); Johannes STOCK, Reuth bei Erbendorf (DE); Andreas BUCHENAUER, Bayreuth (DE); Markus BARTHEL, Kemnath (DE); Konstantin KNODT, Erbendorf (DE); Wolfgang NEUBER, Eschenbach i. d. Opf. (DE); Thomas SCHMAELZLE, Bayreuth (DE); Patrick PLANNERER, Kulmain (DE); Stefan BUCHBINDER, Speichersdorf (DE); Katrin KILIAN, Weiden (DE); Urs HERZOG, Niederrohrdorf (CH)

(73) Assignee: Siemens Healthineers AG, Forchheim (DE)

(21) Appl. No.: 19/547,757

(22) Filed: Feb. 24, 2026

(30) Foreign Application Priority Data
Feb. 25, 2025 (EP) 25159872.8

(51) Int. Cl. A61B 6/04

(52) U.S. Cl. CPC A61B 6/0487; A61B 6/0407

(57) ABSTRACT

One or more example embodiments relates to an apparatus for positioning a patient. The apparatus has a pillar, a patient rest and a hardware module. The hardware module has control electronics for actuating at least one actuator element for changing the spatial position of the patient rest. The hardware module further has a carrier, on which the control electronics are arranged. The hardware module is attached to the pillar such that it can be pivoted about an axis of rotation. The hardware module can be positioned into a first pivot position in which the control electronics are situated in an interior of the pillar and into a second pivot position in which the control electronics are accessible from outside the pillar.

### CROSS-REFERENCE TO RELATED APPLICATION(S)

The present application claims priority under 35 U.S.C. § 119 to European Patent Application No. 25159872.8, filed Feb. 25, 2025, the entire contents of which is incorporated herein by reference.

### FIELD

One or more example embodiments relates to an apparatus for positioning a patient, for example for positioning during radiotherapy via an irradiation system or during generation of image data via an imaging system. One or more example embodiments further relates to a corresponding irradiation system and an imaging system.

### RELATED ART

Apparatuses for positioning a patient, in particular for radiotherapy, radiology, or fluoroscopy, and in particular with adjustable patient rests, are known from the prior art and are referred to inter alia as patient couches, patient tables, or patient supports. The apparatus can serve for example to suitably position a body region of a patient relevant for examination, in particular in a specified target region, for example in a beam path of a radiation source for irradiating the patient, or between an X-ray emitter and an X-ray detector for image acquisition, or in a tube of an MRI device.

For the positioning of the patient, it can be advantageous to be able to vary a position of the body region relevant for examination, for example to be capable of variation with respect to as many spatial directions and/or axes of rotation as possible. To vary the position, actuator elements are known that are mounted in the vicinity of the patient rest and are connected to control electronics, for example. If a number of the spatial directions to be varied and/or axes of rotation to be varied is increased, an installation space requirement of the actuator elements and/or the control electronics increases accordingly. An existing installation space inside the apparatus, for example in the region below the patient rest, is in particular limited and/or is not available for the installation of the increased number of actuator elements and/or the control electronics.

This challenge is overcome in the prior art by making parts of the actuator elements and/or the control electronics decentralized, for example being installed outside the apparatus. Alternatively, the available installation space is increased by enlarging the apparatus, although this is not possible in all use cases. Furthermore, in known solutions, the accessibility to the control electronics, for example, can be restricted.

### SUMMARY

Example embodiments provide an improved concept for integrating control electronics in an apparatus for positioning a patient via which an existing installation space is used more effectively and via which easy access to the control electronics is ensured.

This object is achieved by the independent claim. Advantageous developments and preferred embodiments are the subject matter of the dependent claims.

The improved concept is based on integrating the control electronics inside the apparatus via a pivotable solution.

### BRIEF DESCRIPTION OF THE DRAWINGS

In the following the invention is described in more detail based on specific exemplary embodiments and associated schematic drawings. In the figures identical or functionally identical elements may be provided with the same reference signs. The description of identical or functionally identical elements may not necessarily be repeated in the various figures.

FIG. 1 illustrates a schematic representation of an exemplary embodiment of an irradiation system or an imaging system; and

FIG. 2 illustrates a schematic representation of an exemplary embodiment of an apparatus for positioning a patient; and

FIG. 3 illustrates a schematic representation of a part of a further exemplary embodiment of an apparatus for positioning a patient; and

FIG. 4 illustrates a schematic representation of details of the embodiment from FIG. 2 according to an exemplary embodiment; and

FIG. 5 illustrates a schematic representation of a part of a further exemplary embodiment of an apparatus for positioning a patient; and

FIG. 6 illustrates a schematic representation of a part of a further exemplary embodiment of an apparatus for positioning a patient; and

FIG. 7 illustrates a schematic representation of a further exemplary embodiment of an apparatus for positioning a patient.

### DETAILED DESCRIPTION

According to one or more example embodiments, an apparatus for positioning a patient is presented. The apparatus comprises a pillar, a patient rest—in particular, mounted on the pillar—and a hardware module. The hardware module comprises control electronics for actuating at least one actuator element for changing the spatial position of the patient rest. The hardware module further comprises a carrier, on which the control electronics are arranged. The hardware module, in other words at least the carrier and the control electronics, is attached to the pillar such that it is pivotable about an axis of rotation and the hardware module can be positioned into a first pivot position in which the control electronics are situated in an interior of the pillar. Furthermore, the hardware module can be positioned into a second pivot position in which the control electronics are accessible from outside the pillar.

The patient can be in particular a person on whom an examination and/or a treatment is to be performed. In particular, it should be possible for the patient or at least a part of the patient to be brought into a position that is advantageous for the respective examination and/or treatment. To this end, a change of position of the patient rest is necessary, for example.

In particular, the patient rest can be designed such that the patient can lie, sit or stand on the patient rest. The patient rest can be a reclining surface, a sitting surface, and/or a surface to lean on that can serve to position the patient. The position change of the patient rest can take the form of a change in the position and/or a change in the orientation of the patient rest. In particular, the patient rest can be movably mounted at or on the pillar. In the intended arrangement, the pillar can be located beneath or partially beneath the patient rest, for example.

The pillar can be, for example, a fixed component of the apparatus and can also be referred to as base, pedestal, or substructure of the patient rest, for example. In particular, the pillar can comprise a further interior that can accommodate electrical cables, the at least one actuator element, or further components, for example. The pillar can, for example, comprise at least one support element or consist of the at least one support element in order to hold the patient rest stably in a predefined position. In particular, in the intended arrangement, the pillar can be in contact with an installation area or firmly connected to the installation area.

In particular, the apparatus can additionally comprise the at least one actuator element. The at least one actuator element can be arranged, for example, in an interior of the patient rest or in the interior of the pillar. Likewise, a first actuator element of the at least one actuator element can be situated in the interior of the patient rest and a second actuator element of the at least one actuator element in the interior of the pillar.

The at least one actuator element can also be referred to as at least one actuator. The at least one actuator element can be referred to as a drive unit and can be designed to convert a signal into a mechanical motion, force, pressure, or torque. For example, the at least one actuator element can contain an electric motor, a hydraulic actuator, a pneumatic actuator, a servomotor and/or a cylinder. In particular, the at least one actuator element can contain different types of actuators, in order to initiate the change of position and/or the change of orientation, for example.

The control electronics can contain in particular at least one electronic circuit, in particular a printed circuit board with mounted electronic components, and optionally a housing. The carrier can correspond, for example, to a supporting housing structure that is open on one side for example or comprises at least one further opening. The carrier can also comprise at least one connector plug, cables, and further mounting options. Alternatively, the carrier can also comprise a printed circuit board or consist of a printed circuit board material.

In particular, the carrier is fastened exclusively to the axis of rotation on the pillar. Alternatively, in addition to the fastening to the axis of rotation, the carrier can, for fastening to the pillar, contain a guide rail or a slide rail that is situated for example on a side of the carrier opposing the axis of rotation.

In the intended arrangement, the interior of the pillar can be situated below the patient rest, for example. In particular, the pillar can for example comprise a pillar housing that prevents access to the interior when the hardware module is in the first pivot position. In other words, the access to the hardware module and/or to the control electronics can be restricted or prevented, for example, when the hardware module is in the first pivot position.

The access to the hardware module and/or to the control electronics can make it possible in particular to reach the control electronics with a tool or a hand, for example, in order to connect a data cable for reading and/or transferring data, for example. The access can also mean that components of the control electronics, such as electronic components or plug-in modules, can be inspected, replaced, or repaired. It may be necessary or advantageous for at least a part of the hardware module to be situated outside the pillar. Another part that is located, for example, in the second pivot position in the interior of the pillar can in particular have restricted access since, for example, a guide is necessary in order to carry out the aforementioned steps. In other words, the hardware module can be situated at least partially outside the pillar in the second pivot position so that the control electronics are thus accessible from outside the pillar.

For example, it may be necessary for the purposes of servicing and/or testing and/or repairs to gain access to the control electronics. For these purposes in particular, the hardware module can be positioned from the first into the second pivot position, in particular by pivoting about the axis of rotation, and the control electronics can thus be reached in order to perform a function check or to read out data, for example.

An advantage is that, in the first pivot position, the hardware module can be pivoted into the interior of the pillar and the existing installation space is thus used without the need to increase an external dimension of the apparatus, for example. The access to the control electronics is ensured via the second pivot position and servicing work and/or repair work as well as tests are thus possible in the second pivot position. The apparatus can thus be designed in a compact and space-saving manner with respect to the installation area without needing to compromise on accessibility. A spatial proximity between the control electronics and the at least one actuator element can also be ensured.

Another advantage is, in particular when compared with solutions that provide for translational displaceability of the hardware module in the sense of a drawer or a compartment or the like, that connecting cables that connect the control electronics to components arranged away from the hardware module do not need to be moved as much owing to the rotational movement. This can prevent or reduce unwanted movements or stresses on the connecting cables or corresponding connections of the connecting cables with the control electronics or the hardware module.

According to at least one embodiment, the hardware module comprises a stop element, which is connected to the carrier and which limits a pivot movement of the hardware module from the first pivot position into the second pivot position in the second pivot position.

When the hardware module is in the first pivot position, the stop element can in particular be situated inside the interior of the pillar. During the pivot movement of the hardware module, the stop element can, for example, run in a guide rail or in a detent, for example can be moved with the hardware module. In the second pivot position of the hardware module, the stop element or at least one part of the stop element can, for example, be accessible from outside the pillar. In the second pivot position of the hardware module, the stop element can limit the pivot movement of the hardware module via a locking or an engaging at the pillar, for example.

An advantage of this embodiment is that a movement of the hardware module can be limited to a defined position owing to the stop element. Owing to the limitation by the stop element, the hardware module can move from the first pivot position into the second pivot position and back into the first pivot position. The hardware module and thus the control electronics can thereby be made easily accessible for repair purposes and/or servicing purposes.

According to at least one further embodiment, the stop element can be dismantled non-destructively from the carrier and only when the stop element is dismantled can the hardware module be positioned into a third pivot position in which the control electronics can be dismantled non-destructively from the carrier. Alternatively, the stop element can be released from a detent on the pillar and only when the stop element is released from the detent can the hardware module be positioned into a third pivot position in which the control electronics can be dismantled non-destructively from the carrier.

In particular, the stop element can be dismantled from the carrier and/or released from the detent non-destructively when the hardware module is in the second pivot position. The third pivot position can serve for repair purposes, for example; in particular, the control electronics can be dismantled from the carrier in the third pivot position and/or replaced with other control electronics. In addition, in the third pivot position, additional control electronics can be added to the carrier, for example.

In particular, the dismantled stop element can be mounted in the second pivot position of the hardware module and/or the detent at the pillar can be restored.

An advantage of this embodiment is that, for example when repairing the control electronics, easy access to the hardware module can be ensured by way of the third pivot position. In particular, the third pivot position can improve serviceability of the apparatus for positioning a patient.

According to at least one further embodiment, only when the stop element is dismantled can the hardware module be pivoted from the first pivot position beyond the second pivot position into the third pivot position.

In particular, the hardware module can also be pivotable from the third pivot position beyond the second pivot position into the first pivot position.

An advantage of this embodiment is that the hardware module is in a defined position in each case. The first pivot position and the second pivot position can be reached without dismantling the stop element. The third pivot position can, for example, be made accessible only to trained service personnel via the necessary dismantling of the stop element.

According to at least one further embodiment, the apparatus comprises a cover that can be mounted on the pillar when the hardware module is in the first pivot position. Furthermore, only when the cover is dismantled can the hardware module be pivoted from the first pivot position into the second pivot position.

In particular, the hardware module is not accessible from outside the interior of the pillar when the cover is mounted on the pillar. In particular, the hardware module is covered by the cover and is thus protected against environmental influences such as dust and/or moisture, for example. Furthermore, the hardware module can be protected from unauthorized access. In particular, the cover cannot be mounted when the hardware module is in the second pivot position or in the third pivot position. In other words, the hardware module is forced into the first pivot position, for example, by mounting the cover on the pillar.

An advantage of this embodiment is that mounting the cover protects the hardware module against environmental influences. The hardware module can also be protected from unauthorized access via the mounted cover. In addition, an aesthetic advantage can be obtained by integrating the cover in the pillar.

According to at least one further embodiment, the apparatus comprises a cover that is connected to the hardware module and that can be pivoted together with the hardware module from the first pivot position into the second pivot position.

In the first pivot position, the cover can cover the hardware module and thus protect the hardware module against environmental influences and unauthorized access. In particular, when the stop element is dismantled, the cover can be pivoted together with the hardware module from the second pivot position into the third pivot position. In particular, the cover can be connected to the carrier, for example via a threaded connection. In particular, the cover can alternatively be part of the carrier.

An advantage of this embodiment is that the hardware module can be pivoted from the first pivot position into the second pivot position without the need to dismantle the cover. This can result in quick and easy serviceability of the hardware module.

According to at least one further embodiment, the apparatus comprises a further cover that is connected to the pillar and is aligned with the cover when the hardware module is in the first pivot position.

The cover and/or the further cover can essentially comprise flat surfaces, for example. The further cover and the cover can be designed to be flush, for example, when the hardware module is in the first pivot position. In particular, the cover is mounted on the pillar.

An advantage of this embodiment is that the interior of the pillar can be protected against environmental influences and/or unauthorized access when the cover and the further cover are aligned. In addition, the described embodiments can result in an aesthetic advantage for the apparatus.

According to at least one further embodiment, the axis of rotation, in particular in the intended arrangement of the apparatus, runs along a vertical direction of the apparatus.

In particular, the pivot movement of the hardware module from the first pivot position into the second pivot position can, in the intended arrangement of the apparatus, run in a horizontal plane perpendicular to the vertical direction of the apparatus. In particular, the pivot movement from the second pivot position into the third pivot position can also run in the horizontal plane of the apparatus.

An advantage of this embodiment is that the pivot movement of the hardware module requires little force, for example, because in particular no movement is required in the vertical direction. The pivot movement is thus less dependent on a weight of the hardware module.

According to at least one further embodiment, the hardware module comprises an electrical connecting cable for connecting the control electronics to the at least one actuator element. Furthermore, the electrical connecting cable runs from the axis of rotation along a surface of the carrier to the control electronics.

The electrical connecting cable can run, for example, on a side of the hardware module that faces the interior of the pillar when the hardware module is in the first pivot position. In particular, the electrical connecting cable can furthermore run inside the interior of the pillar. For example, the carrier has a rectangular form and the electrical connecting cable runs on a short side of the carrier in the region of the axis of rotation. The axis of rotation can be situated at an end of the short side of the carrier, in other words in a corner of the rectangular shape. In particular, a mechanical stress or blockage or movement of the electrical connecting cable during the pivot movement from the first pivot position into the second pivot position is prevented or reduced by the spatial proximity to the axis of rotation.

In particular, the hardware module can comprise a hollow axle. For example, the axis of rotation can run through the hollow axle, for example through a center of the hollow axle. The hollow axle can at least partially enclose the electrical connecting cable, for example.

An advantage of this embodiment is that unwanted movements and/or stresses on the electrical connecting cable due to the rotational movement of the hardware module can be prevented or reduced. To this end, the electrical connecting cable can run in a spatial proximity to the axis of rotation and/or can run through the hollow axle. In particular, the electrical connecting cable therefore need not be detached from the control electronics nor from the at least one actuator element, for example, when the hardware module moves from the first pivot position into the second pivot position.

According to at least one further embodiment, the apparatus comprises the at least one actuator element.

According to at least one further embodiment, the at least one actuator element is designed to move the patient rest according to at least one translational axis and/or according to at least one rotational axis.

In particular, the at least one actuator element can be situated inside the pillar and/or inside or on the patient rest. For example, the at least one actuator element can be designed to move the patient rest according to three translational axes and/or according to three rotational axes. In particular, in the intended arrangement, the pillar can remain in its position during a movement of the patient rest.

An advantage of this embodiment is that a flexible change in position of the patient rest is possible, and the patient situated on the patient rest can thus be moved into a position that is advantageous for the respective examination and/or treatment. In particular, a position of the patient relative to the patient rest can remain unchanged.

According to at least one further embodiment, the pillar comprises a base frame located on an underside of the apparatus. Furthermore, the hardware module is attached to the base frame such that it can be pivoted about the axis of rotation.

In particular, in the intended arrangement, the underside of the apparatus can be in contact with the installation surface of the apparatus, in other words in particular a building floor and/or a floor of a treatment room or an examination room. For example, the base frame can be screwed onto the installation surface. In particular, the hardware module is only attached to the base frame and is not additionally attached to another component of the pillar.

In particular, the patient rest can be situated on a top side of the apparatus, with the top side being located opposite the underside.

An advantage of this embodiment is that the hardware module is spatially distant from the patient rest. Therefore, for example, an influence on the control electronics by electromagnetic radiation directed toward the patient rest can be reduced. This means that, for example, unwanted impairment or damage of the control electronics by the electromagnetic radiation and/or ionizing radiation can be avoided. A function of an irradiation system or an imaging system due to the control electronics, in particular due to high-frequency signals generated by the control electronics, can also be prevented.

According to at least one further embodiment, the apparatus comprises an electromagnetic shielding apparatus, wherein the hardware module in the first pivot position is on the first side of the electromagnetic shielding apparatus and the patient rest is on a second side of the electromagnetic shielding apparatus, the second side being opposite the first side.

For example, the apparatus can be exposed to electromagnetic radiation through the use of an irradiation unit or an imaging unit, in particular when the hardware module is in the first pivot position. The electromagnetic shielding apparatus can, for example, be integrated inside the interior of the pillar. The electromagnetic shielding apparatus can also be part of the base frame.

An advantage of this embodiment is that the hardware module, in particular the control electronics, can be better protected against the electromagnetic radiation owing to the electromagnetic shielding apparatus. In addition, an influence on the irradiation unit and/or the imaging unit by the control electronics can be further reduced by the electromagnetic shielding apparatus. On the one hand, this can improve a quality of the radiation therapy and/or the generated image data, and on the other hand this can increase a service life of the control electronics.

According to at least one further embodiment, the electromagnetic shielding apparatus contains a metal plate, for example a steel plate.

In particular, in the intended arrangement, the metal plate can be arranged in the base frame or connected to the base frame.

An advantage of this embodiment is that the metal plate can have a high electromagnetic shielding effect. At the same time, a weight of the metal plate can have an advantageous effect on a stability of the apparatus, in particular during a position change of the patient rest by the at least one actuator element.

According to one or more example embodiments, an irradiation system is presented. The irradiation system comprises an irradiation device for generating ionizing radiation and an apparatus for positioning a patient according to one or more example embodiments.

According to at least one embodiment, the irradiation device can be designed to generate a gamma radiation and/or an X-ray radiation and/or a neutron radiation and/or a proton radiation and/or a heavy ion radiation and/or an electron radiation.

According to one or more example embodiments, an imaging system is presented. The imaging system comprises an imaging unit for generating image data and an apparatus for positioning a patient according one or more example embodiments.

According to at least one embodiment, the imaging unit corresponds to an X-ray based imaging unit and/or a positron emission tomography imaging unit (PET imaging unit) and/or a magnetic resonance imaging unit (MRI unit).

In particular, the X-ray based imaging unit can correspond to a computed tomography imaging unit (CT imaging unit) and/or a C-arm machine and/or a classic X-ray device.

Further embodiments of the imaging system according to the invention result directly from the various designs of the apparatus according to the invention. In particular, individual features and corresponding explanations and advantages relating to the various embodiments of the apparatus according to the invention may be transferred analogously to corresponding embodiments of the imaging system according to the invention.

Further features and combinations of features of the invention result from the figures and the description of these and from the claims. In particular, further embodiments of the invention do not necessarily have to contain all features of one of the claims. Further embodiments of the inventions may have features and combinations of features not mentioned in the claims.

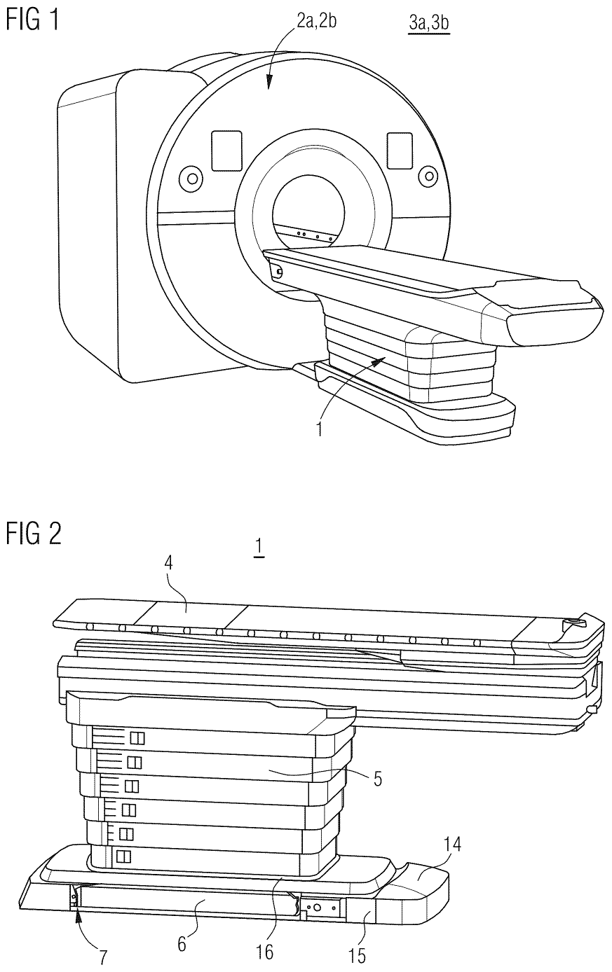

FIG. 1 shows a schematic representation of an exemplary embodiment of an irradiation system 3a or an imaging system 3b. The irradiation system 3a contains an irradiation device 2a or the imaging system 3b contains an imaging unit 2b. Both the irradiation system 3a and the imaging system 3b contain an apparatus 1 for positioning a patient. In particular, in the intended arrangement, the apparatus 1 for positioning the patient can be arranged immediately in front of the irradiation device 2a or the imaging unit 2b. In particular, for irradiating the patient and/or for generating image data, a part of the apparatus 1, for example a patient rest 4 (see FIG. 2), can be located inside an interior of the imaging unit 2b.

The irradiation device 2a can be designed, for example, to generate a gamma radiation, an X-ray radiation, a neutron radiation, a proton radiation, a heavy ion radiation, and/or an electron radiation.

The imaging unit 2b can, for example, contain a CT imaging unit, an MRI unit, a PET imaging unit, a C-arm imaging unit, or a fluoroscopy imaging unit, or can correspond thereto.

In particular, in addition to the irradiation device 2a, the irradiation system 3a can also contain an imaging unit 2b and thus be designed to generate radiation for an irradiation therapy via the irradiation device 2a as well as being designed to generate the image data via the imaging unit 2b.

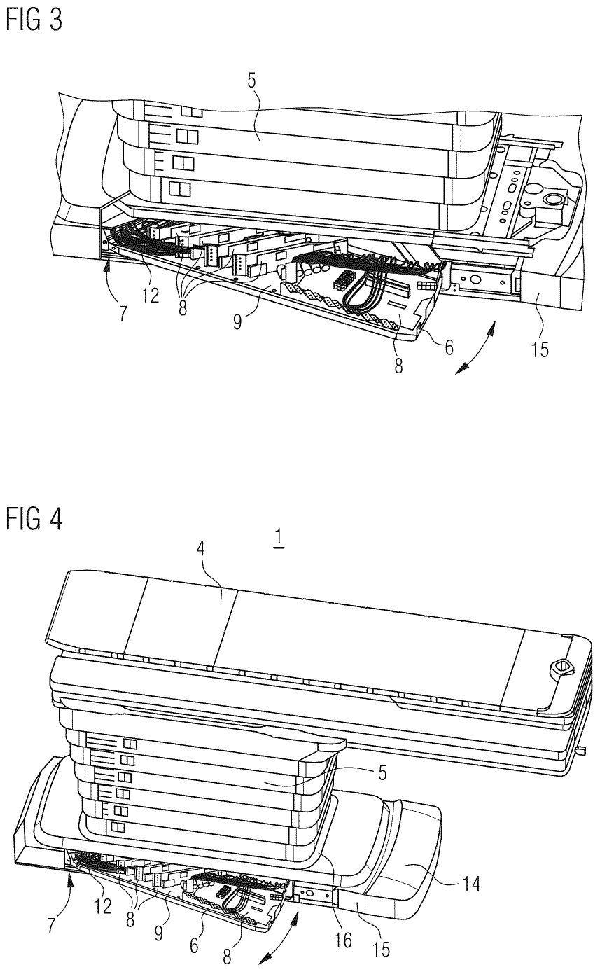

FIG. 2 shows a schematic representation of an exemplary embodiment of an apparatus 1 for positioning a patient. The apparatus 1 comprises a pillar 5, the patient rest 4 and a hardware module 6. The hardware module 6 comprises control electronics 8 (see FIG. 3) for actuating at least one actuator element (not shown) for changing the spatial position of the patient rest 4. The hardware module 6 is attached to the pillar 5 such that it can be pivoted about an axis of rotation 7.

Here, the hardware module 6 can be positioned into a first pivot position in which the control electronics 8 are situated in an interior of the pillar 5. Furthermore, the hardware module 6 can be positioned into a second pivot position in which the control electronics 8 are accessible from outside the pillar. The representation in FIG. 2 corresponds to the first pivot position of the hardware module 6. In other words, example embodiments implement a two-stage solution with respect to the first and second pivot position of the hardware module 6.

In a further embodiment, the pillar 5 can comprise a base frame 14 located on the underside of the apparatus 1. In particular, in the intended arrangement, the base frame 14 can be connected to an installation surface, for example via a threaded connection or a detent, in order to stabilize the apparatus 1. In particular, in the intended arrangement, the pillar 5 can be stationary with respect to the installation surface and only the position of the patient rest 4 can be changed.

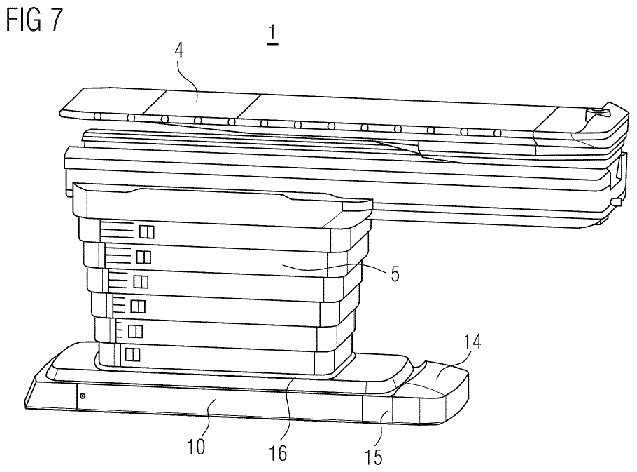

In particular, the apparatus 1 can comprise a cover 10 (see FIG. 7) that can be mounted on the pillar 5 when the hardware module 6 is in the first pivot position. In particular, only when the cover 10 is dismantled can the hardware module 6 be pivoted from the first pivot position into the second pivot position. FIG. 2 shows an exemplary representation of the apparatus 1 with the cover 10 dismantled.

The apparatus 1 can furthermore comprise a further cover 15 that is connected to the pillar 5 and is aligned with the cover 10 when the hardware module 6 is in the first pivot position. This configuration is shown in particular in FIG. 7.

The apparatus 1 can comprise an electromagnetic shielding apparatus 16, the hardware module 6 in the first pivot position being on a first side of the electromagnetic shielding apparatus 16 and the patient rest 4 being on a second side of the electromagnetic shielding apparatus 16, the second side being opposite the first side.

The electromagnetic shielding apparatus 16 can, for example, be located on a top side of the base frame 14, the hardware module 6 being arranged, for example, in an interior of the base frame 14. The electromagnetic shielding apparatus 16 can, for example, protect the control electronics 8 against environmental influences, in particular due to electromagnetic radiation, for example caused by the irradiation device 2a or the imaging unit 2b. On the other hand, the electromagnetic shielding apparatus 16 can also prevent a generation of artifacts or a reduction in the signal-to-noise ratio during the generation of image data by shielding an emitted electromagnetic field of the control electronics 8 from a receiving unit of the imaging unit 2b. In particular, it can be advantageous for the electromagnetic shielding apparatus 16 to be located in the immediate vicinity of, for example above, the control electronics 8.

The electromagnetic shielding apparatus 16 can contain a metal plate, for example a steel plate, or can correspond to the metal plate. In particular, the metal plate can advantageously contribute to the stability of the apparatus 1 when the metal plate is situated in a region near the installation surface and thus causes a center of gravity of the apparatus 1 in a lower region of the apparatus 1.

FIG. 3 shows a schematic representation of a part of a further exemplary embodiment of the apparatus 1. A pivot movement is illustrated by an arrow along the hardware module 6. In particular, the pivot movement can take the form of a pivoting from the first pivot position into the second pivot position or vice versa.

The control electronics 8 can comprise at least one electronic circuit. FIG. 3 shows a plurality of electronic circuits on at least one printed circuit board. The representation in FIG. 3 can correspond, for example, to the second pivot position or to an intermediate position between the first pivot position and the second pivot position.

In particular, the hardware module 6 can comprise an electrical connecting cable 12 for connecting the control electronics 8 to the at least one actuator element. The electrical connecting cable 12 runs from the axis of rotation along a surface of the carrier 9 to the control electronics 8. In particular, the electrical connecting cable 12 runs in a region of the axis of rotation 7. At least one part of the electrical connecting cable 12, which part is situated at a transition from the interior of the pillar 5 to the hardware module 6, can run in the immediate vicinity of the axis of rotation 7. The electrical connecting cable 12 can run further into the interior of the pillar 5 and be guided there to each of the at least one actuator element.

FIG. 4 shows a schematic representation of the exemplary embodiment from FIG. 2. In this representation, the hardware module is shown in the second pivot position. Unless otherwise described, a part of the explanations or all explanations from FIG. 2 and FIG. 3 apply to this embodiment. The pivot movement is illustrated by an arrow along the hardware module 6.

FIG. 5 shows a schematic representation of a part of a further exemplary embodiment of an apparatus 1. In this representation, the hardware module 6 is in the first pivot position.

The hardware module 6 can comprise a stop element 11, which is connected to the carrier 9 and which limits the pivot movement of the hardware module 6 from the first pivot position into the second pivot position in the second pivot position.

The stop element 11 can run, for example, in a guide rail or in a detent arranged in the interior of the pillar 5. In particular in the first pivot position, the stop element 11 cannot be accessed from an exterior of the pillar 5. In particular, the stop element 11 can be dismantled from the carrier 9 and/or released from the guide rail or from the detent non-destructively.

In particular, the representation in FIG. 5 shows a guide of the electrical connecting cable 12 from the interior of the pillar 5 through a circular opening, which is also arranged in the interior of the pillar 5, to the hardware module 6, in particular on the carrier 9.

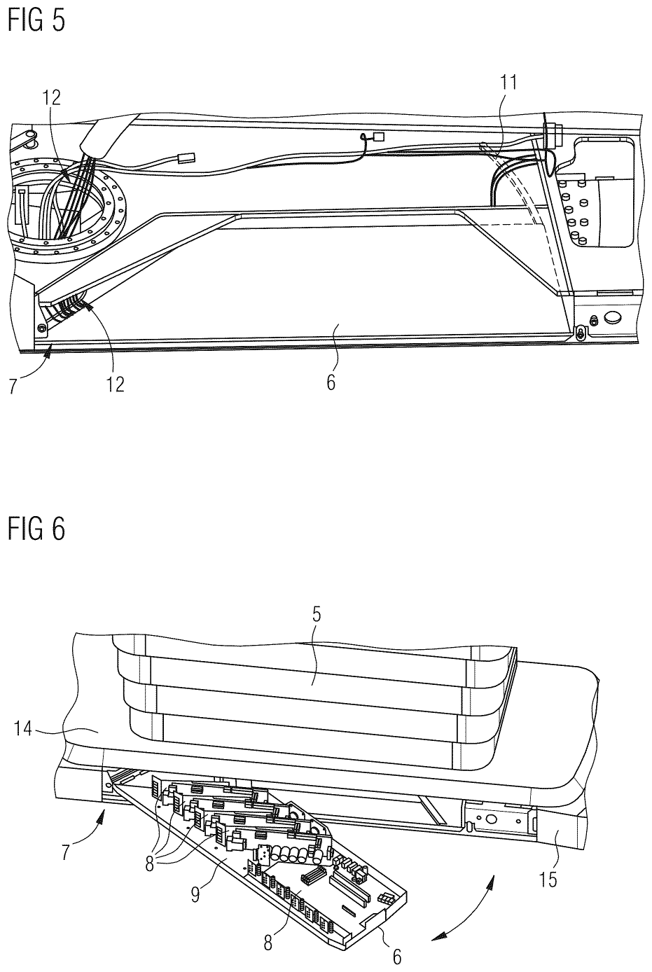

FIG. 6 shows a schematic representation of a part of a further exemplary embodiment of an apparatus 1. In comparison to FIG. 5, the stop element 11 is not shown in the representation in FIG. 6, because, for example, it is dismantled. For example, only when the stop element 11 is dismantled can the hardware module 6 be positioned in a third pivot position in which the control electronics 8 can be dismantled non-destructively from the carrier 9. In the representation in FIG. 6, the hardware module 6 is in the third pivot position. In other words, a three-stage solution can thus be implemented with respect to the first, second, and third pivot position of the hardware module 6. The pivot movement is illustrated by an arrow along the hardware module 6.

For example, the hardware module 6 can be pivoted from the first pivot position beyond the second pivot position into the third pivot position when the stop element 11 is dismantled. Also, the hardware module 6 can be pivoted from the third pivot position beyond the second pivot position into the first pivot position. In addition, in some embodiments, the hardware module 6 can be further pivoted beyond the first pivot position into the interior of the pillar 5. In the second pivot position, the stop element 11 can be mounted, for example, so that the pivot movement of the hardware module 6 can thus be limited.

FIG. 7 shows a schematic representation of a further exemplary embodiment of the apparatus 1. In this representation, the hardware module 6 is shown in the first pivot position. Unless otherwise described, a part of the explanations or all explanations from the other representations apply to this embodiment.

In particular, the representation shows the cover 10, which is mounted on the pillar 5 and is aligned with the further cover 15. Also shown is a possible arrangement of the electromagnetic shielding apparatus 16, which is situated above the hardware module 6 on the base frame 14.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. An apparatus for positioning a patient, the apparatus comprising:
a pillar;
a patient rest; and
a hardware module including,
control electronics configured to actuate at least one actuator element for changing a spatial position of the patient rest, and
a carrier, the control electronics being on the carrier, wherein
the hardware module is attached to the pillar such that the hardware module is pivotable about an axis of rotation,
the hardware module is positionable into a first pivot position in which the control electronics are in an interior of the pillar, and
the hardware module is positionable into a second pivot position in which the control electronics are accessible from outside the pillar.

2. The apparatus of claim 1, wherein the hardware module comprises a stop element connected to the carrier, the stop element limits a pivot movement of the hardware module from the first pivot position into the second pivot position in the second pivot position.

3. The apparatus of claim 2, wherein
the hardware module is positionable into a third pivot position in which the control electronics is dismantable non-destructively from the carrier only when the stop element is dismantled from the carrier; or
the hardware module is positionable into a third pivot position in which the control electronics is dismantable non-destructively from the carrier only when the stop element is released from a detent on the pillar.

4. The apparatus of claim 3, wherein
the hardware module is pivotable from the first pivot position beyond the second pivot position into the third pivot position only when the stop element is dismantled; or
the hardware module is pivotable from the first pivot position beyond the second pivot position into the third pivot position only when the stop element is released from the detent.

5. The apparatus of claim 1, further comprising:
a cover, the cover being mountable on the pillar when the hardware module is in the first pivot position, wherein
the hardware module is pivotable from the first pivot position into the second pivot position only when the cover is dismantled.

6. The apparatus of claim 1, further comprising:
a cover connected to the hardware module, the cover and the hardware module being pivotable together from the first pivot position into the second pivot position.

7. The apparatus of claim 5, further comprising:
a further cover connected to the pillar and aligned with the cover when the hardware module is in the first pivot position.

8. The apparatus of claim 1, wherein the axis of rotation extends along a vertical direction of the apparatus.

9. The apparatus of claim 1, wherein
the hardware module includes an electrical connecting cable to connect the control electronics to the at least one actuator element; and
the electrical connecting cable extends from the axis of rotation along a surface of the carrier to the control electronics.

10. The apparatus of claim 1, wherein
the apparatus includes the at least one actuator element; and
the at least one actuator element is configured to move the patient rest according to at least one of at least one translational axis or at least one rotational axis.

11. The apparatus of claim 1, wherein
the pillar includes a base frame on an underside of the apparatus; and
the hardware module is attached to the base frame such that the hardware module is pivotable about the axis of rotation.

12. The apparatus of claim 11, further comprising:
an electromagnetic shielding apparatus, wherein the hardware module in the first pivot position is on a first side of the electromagnetic shielding apparatus and the patient rest is on a second side of the electromagnetic shielding apparatus opposite the first side.

13. The apparatus of claim 12, wherein the electromagnetic shielding apparatus contains a metal plate.

14. An irradiation system, comprising:
an irradiation device configured to generate ionizing radiation; and
an apparatus including,
a pillar,
a patient rest, and
a hardware module including,
control electronics configured to actuate at least one actuator element for changing a spatial position of the patient rest, and
a carrier, the control electronics being on the carrier, wherein
the hardware module is attached to the pillar such that the hardware module is pivotable about an axis of rotation,
the hardware module is positionable into a first pivot position in which the control electronics are in an interior of the pillar, and
the hardware module is positionable into a second pivot position in which the control electronics are accessible from outside the pillar.

15. An imaging system, comprising:
an imaging unit configured to generate image data; and
an apparatus including,
a pillar,
a patient rest, and
a hardware module including,
control electronics configured to actuate at least one actuator element for changing a spatial position of the patient rest, and
a carrier, the control electronics being on the carrier, wherein
the hardware module is attached to the pillar such that the hardware module is pivotable about an axis of rotation,
the hardware module is positionable into a first pivot position in which the control electronics are in an interior of the pillar, and
the hardware module is positionable into a second pivot position in which the control electronics are accessible from outside the pillar.

16. The apparatus of claim 2, further comprising:
a cover, the cover being mountable on the pillar when the hardware module is in the first pivot position, wherein
the hardware module is pivotable from the first pivot position into the second pivot position only when the cover is dismantled.

17. The apparatus of claim 16, further comprising:
a cover connected to the hardware module, the cover and the hardware module being pivotable together from the first pivot position into the second pivot position.

18. The apparatus of claim 17, further comprising:
a further cover connected to the pillar and aligned with the cover when the hardware module is in the first pivot position.

19. The apparatus of claim 2, wherein
the apparatus includes the at least one actuator element; and
the at least one actuator element is configured to move the patient rest according to at least one of at least one translational axis or at least one rotational axis.

20. The apparatus of claim 2, wherein
the pillar includes a base frame on an underside of the apparatus; and
the hardware module is attached to the base frame such that the hardware module is pivotable about the axis of rotation.
